(19) United States
(12) Patent Application Publication
(10) Pub. No.: US 2012/0288935 A1
(43) Pub. Date: Nov. 15, 2012

(54) "Click" Nanoparticle Conjugates

(75) Inventors: Chad A. Mirkin, Wilmette, IL (US); Joshua I. Cutler, Somerville, NJ (US)

(73) Assignee: NORTHWESTERN UNIVERSITY, Evanston, IL (US)

(21) Appl. No.: 13/497,214

(22) PCT Filed: Sep. 22, 2010
(86) PCT No.: PCT/US2010/049782

§ 371 Date: Aug. 7, 2012

Related U.S. Application Data
(60) Provisional application No. 61/244,918, filed on Sep. 23, 2009.
(60) Provisional application No. 61/288,378, filed on Dec. 21, 2009.
(60) Provisional application No. 61/315,441, filed on Mar. 19, 2010.

(51) Int. Cl. C07F 15/02; C12N 5/071; B82Y 5/00

(52) U.S. Cl. 435/375; 536/23.1; 530/395; 530/391.3; 428/402; 977/906; 977/774

(57) ABSTRACT

Modified nanoparticles are disclosed. More specifically, nanoparticles modified with an agent through a triazole linkage are disclosed. Also disclosed are methods of preparing modified nanoparticles and methods of using these modified nanoparticles.

### CROSS REFERENCE TO RELATED APPLICATIONS

This application claims the benefit of U.S. provisional application No. 61/244,918, filed Sep. 23, 2009, U.S. provisional application No. 61/288,378, filed Dec. 21, 2009; and U.S. provisional application No. 61/315,441, filed Mar. 19, 2010, the disclosures of which are each incorporated by reference in its entirety herein.

### STATEMENT OF U.S. GOVERNMENT INTEREST

This invention was made with U.S. government support under Grant No. 5DPI OD 000285 awarded by the National Institutes of Health, and Grant Number 1U54CA119341 awarded by the National Institutes of Health (NIH)/National Cancer Institute/Centers of Cancer Nanotechnology Excellence (NCI/CCNE). The government has certain rights in the invention.

### BACKGROUND

Polyvalent nucleic acid gold nanoparticle conjugates are a unique class of hybrid bio-nanomaterials formed by functionalizing gold nanoparticles, typically 2-250 nm in diameter, with a dense oligonucleotide shell. The ability to generate such structures with high surface densities of oligonucleotides (about 2×10¹³ oligos/cm²) has led to the discovery and subsequent study of many fundamentally new properties, including cooperative melting transitions (1, 2), enhanced affinities for complementary oligonucleotides (3, 4), hybridization dependent optical responses (5), enhanced catalytic behavior (6), resistance to enzymatic degradation (7), and high cellular uptake without the need for transfection agents (8). These conjugate properties have led to many important applications in several areas of research, including programmable colloidal assembly and crystallization (9, 10), gene regulation (8), and high sensitivity metal ion and molecular diagnostics (11-13), some of which have been commercialized and recently FDA approved.

Although there have been attempts to extend such chemistry to other particle compositions, including silver (14), semiconductor quantum dots (15), silica (16), and other oxides (17), the thiol adsorption on gold chemistry still stands as one of the most versatile ways of making stable conjugates with tailorable oligonucleotide surface compositions and densities. New chemistry is needed for broadening the scope of inorganic nanomaterial conjugates that exhibit the aforementioned properties unique to the polyvalent nucleic acid gold nanoparticle conjugates.

### SUMMARY

Disclosed herein are nanoparticles having an agent attached to their surfaces. More specifically, disclosed herein are nanoparticles modified with agents on their surfaces through a triazoyl group.

In some aspects, the disclosed nanoparticles have the agent modified on their surfaces with a surface density of at least 5 pmol/cm². In some cases, the surface density of the agent is at least 10 pmol/cm². The disclosed nanoparticles can be magnetic or paramagnetic. In some specific embodiments, the nanoparticle can comprise iron oxide.

In various embodiments, the agent comprises an oligonucleotide, a protein, a peptide, an antibody, a non-peptide drug, or mixtures thereof. In some embodiments, when the agent comprises an oligonucleotide, the oligonucleotide can be a DNA oligonucleotide and/or RNA oligonucleotide. The oligonucleotide can comprise 5 to 200 nucleobases. In various cases, the oligonucleotide can be a peptide nucleic acid. In various cases, the oligonucleotide can comprise at least one modified internucleoside linkage selected from the group consisting of phosphorothioate linkage, a morpholino linkage, a methylphosphonate linkage, and a sulfonyl linkage. The oligonucleotide can be bound to the nanoparticle through a 5′ linkage or a 3′ linkage. In some cases, the oligonucleotide can be bound to the nanoparticle through a spacer. In some specific cases, the spacer is a polymer, such as a water-soluble polymer, nucleic acid, polypeptide, and/or oligosaccharide.

In some embodiments, the oligonucleotide is complementary to all or a portion of polynucleotide encoding for a gene product. The oligonucleotide can be 100% complementary to the polynucleotide, greater than 95% complementary to the polynucleotide, greater than 90% complementary to the polynucleotide, greater than 80% complementary to the polynucleotide, greater than 75% complementary to the polynucleotide, greater than 70% complementary to the polynucleotide, greater than 65% complementary to the polynucleotide, greater than 60% complementary to the polynucleotide, greater than 55% complementary to the polynucleotide, or greater than 50% complementary to the polynucleotide. In various cases, the oligonucleotide is complementary to a coding region of the polynucleotide. In some cases, the oligonucleotide is complementary to a non-coding region of the polynucleotide. The polynucleotide can be bacterial (such as bacterial genomic DNA, RNA transcribed from bacterial genomic DNA); viral (such as viral genomic RNA, viral genomic DNA, RNA transcribed from viral genomic DNA); or fungal (such as fungal genomic DNA, RNA transcribed from fungal genomic DNA).

In various aspects, the disclosed nanoparticle can further comprise a second agent. In some embodiments, the second agent comprises an oligonucleotide, a protein, a peptide, an antibody, a non-peptide drug, or combinations thereof. In some specific embodiments, the second agent comprises an oligonucleotide having 5 to 200 nucleobases.

In various aspects, the disclosed nanoparticle can further comprise a label. The label can comprise a fluorophore. In various cases, the fluorophore is covalently attached to the agent. In some cases, the fluorophore is covalently attached to the nanoparticle. The fluorophore can be attached to the nanoparticle through a spacer. In various cases, the spacer comprises a polymer, such as a water soluble polymer. In some specific cases, the polymer comprises an oligonucleotide, an oligosaccharide, or a polyethylene glycol. In some cases, the fluorophore can be selected from the group consisting of a fluorescein dye, 6-((7-amino-4-methylcoumarin-3-acetyl)amino)hexanoic acid, 5(and 6)-carboxy-X-rhodamine, a rhodamine dye, a benzophenoxazine, Cyanine 2 (Cy2) dye, Cyanine 3 (Cy3) dye, Cyanine 3.5 (Cy3.5) dye, Cyanine 5 (Cy5) dye, Cyanine 5.5 (Cy5.5) dye, Cyanine 7 (Cy7) dye, Cyanine 9 (Cy9) dye, 6-carboxy-4′,5′-dichloro-2′,7′-dimethoxyfluorescein, and 5(6)-carboxy-tetramethyl rhodamine.

Further disclosed herein is a method of preparing a modified nanoparticle comprising admixing a nanoparticle having an azide group on at least a portion of the nanoparticle surface, an agent having an alkyne group, a copper (II) salt, a reducing agent, and a copper ligand to form the nanoparticle modified with the agent having a triazolyl linkage between the nanoparticle and the agent. In some cases, the reducing agent comprises ascorbic acid. In various cases, the copper ligand comprises a tris-triazole group. In some specific cases, the agent is an oligonucleotide, a protein, a peptide, an antibody, a non-peptide drug, or combinations thereof. In various cases, the method can further comprise admixing a nanoparticle having an amine group on at least a portion of the nanoparticle surface and a carboxylic acid or activated acid having a azide group to form an amide bond and to provide the nanoparticle having an azide group. The activated acid can comprise a succinimidyl group, such as succinimidyl ester of a N₃—C₁-C₁₀alkyl carboxylic acid.

Also disclosed herein is a method of preparing a modified nanoparticle as disclosed herein comprising admixing a nanoparticle having an azide group on at least a portion of the nanoparticle surface, an agent having an alkyne group, a copper (II) salt, a reducing agent, and a copper ligand in an organic-aqueous two phase system under conditions sufficient to form the nanoparticle modified with the agent having a triazolyl linkage between the nanoparticle and the agent. In some cases, the method further comprises separating the two phase system to obtain the nanoparticles modified with the agent in the aqueous phase.

Yet another aspect disclosed herein is a method of delivering an agent to a cell comprising contacting the cell with a nanoparticle as disclosed herein. In some cases, the agent comprises a diagnostic agent. In various cases, the agent comprises a therapeutic agent. The cell can be in vivo. The cell can be in vitro. The cell can be mammalian. The cell can be human. The cell can be a cancer cell. The cancer cell can be selected from the group consisting of esophageal, hepatocellular, skin, bladder, bronchogenic, colon, colorectal, gastric, lung, small cell carcinoma, non-small cell carcinoma of the lung, adrenocortical, thyroid, pancreatic, breast, ovarian, prostate, adenocarcinoma, sweat gland, sebaceous gland, papillary carcinoma, papillary adenocarcinoma, cystadenocarcinoma, medullary, renal cell, bile duct carcinoma, choriocarcinoma, seminoma, embryonal, Wilm's tumor, cervical, uterine, testicular, osteogenic, epithelieal, and nasopharyngeal cancer cells.

### BRIEF DESCRIPTION OF THE FIGURES

FIG. 1A shows Melting transitions for DNA-SPION aggregates (10 nm in diameter) at various salt concentrations: 0.15, 0.30, 0.50, and 0.70 M. FIG. 1B is a plot of Tₙ, as a function of salt concentration. FIG. 1C is an example of the derivative of a melting curve for DNA-SPION conjugates with a FWHM of 2° C.

FIG. 2A shows melting transitions for DNA-SPION aggregates (10 nm in diameter) with various loadings. FIG. 2B is a plot of Tₘ as a function of DNA strands/particles. FIG. 2C is a plot of FWHM of melting transitions as a function of DNA strands/particle.

FIG. 3A shows an ICP analysis of modified nanoparticles, indicating that these particles are taken up into HeLa cells in higher concentrations than the unmodified counterparts. FIG. 3B shows fluorescence microscopy image of HeLa cells incubated with modified nanoparticles for 24 h. The fluorescence indicates that the cells took up DNA-SPIONs labeled with Cy5 and were located mainly in the endocytotic vesicles. Scale bar is 30 μm.

### DETAILED DESCRIPTION

Superparamagnetic iron oxide nanoparticles (SPIONs) or other nanoparticles functionalized with azides can be rapidly coupled to alkyne-modified agents, such as oligonucleotides or other biomolecules, to create stable polyvalent conjugates with exceptionally high surface densities of the agent. Alternatively, the nanoparticles are functionalized with alkynes that can be rapidly coupled to azide-modified agents.

This method affords nanoparticles that exhibit properties such as sharp melting transitions and high cellular uptake, indicative of their high surface density functionalization of the agent. The ability to densely functionalize nanoparticles, such as SPIONs, with various agents, including DNA, allows a myriad of applications, such as magnetic resonance imaging (MRI) imaging, magnetic hyperthermia therapy strategies, and assembly of magnetic structures for electronic memory applications (29). In addition, click chemistry can be used as a general strategy for the addition of an agent to nanoparticles and provide modified nanoparticles having surface densities of at least 2 pmol/cm², regardless of core material. The high heat of formation of the triazolyl group in the click reaction described herein allows for higher surface densities that are otherwise unachievable through other coupling reactions. In fact, prior reports of modification of a iron oxide nanoparticle with an oligonucleotide through a linkage other than an azole, resulted in broad melting transitions, not suitable for a majority of diagnostic and detection assays which employ oligonucleotide-modified nanoparticles (see, e.g., Jin et al., J. Am. Chem. Soc., 125:1643-1654 (2003)). Furthermore, the high moiety specificity of the click chemistry reaction described here, e.g., the reaction occurs between an alkyne and azide only, allows for little or no other side reaction or coupling to occur, providing control of the composition of the resulting modified nanoparticle.

The copper(I)-catalyzed azide-alkyne cycloaddition click reaction (18) has been recognized as a facile and versatile chemistry for bioconjugation (19), and has thus garnered significant interest in the field of nanotechnology due to its ability to effectively couple materials together (20-24). Click chemistry is a functional group tolerant reaction that forms triazole linkages under a vast array of conditions (18). For conjugation of an agent to nanoparticles, this reaction is an attractive choice due its high functional group tolerance. For cases where the agent comprises an oligonucleotide, this reaction is further attractive due to its bioorthogonality and high heat of formation, which can promote conjugation in a high salt environment required to overcome the coulombic repulsion of neighboring oligonucleotides. It has been shown that azide-functionalized gold nanoparticles can be assembled in a linear fashion along the backbone of alkyne modified double helices of DNA (25), but without the goal of assembling DNA on to the nanoparticle surface to create high density DNA-nanostructure hybrids. Because a click chemistry reaction is compatible with DNA (21, 26), nanostructures other than those based upon gold can be synthesized using click chemistry and containing a dense monolayer of oligonucleotides or other agents.

Nanoparticles as provided herein have a packing density of the agent on the surface of the nanoparticle that is, in various aspects, sufficient to result in cooperative behavior between nanoparticles and between oligonucleotide or other agents on a single nanoparticle. Agents, and in particular oligonucleotides, have high steric and electronic repulsion to each other, which impedes high packing density (surface density) on the agent, biomolecule, or oligonucleotide on the nanoparticle surface. The use of click chemistry provides nanoparticles modified on their surfaces with agents (e.g., oligonucleotide) at a surface density that is not achievable using other modification methods.

The cooperative behavior between the nanoparticles increases the resistance of the oligonucleotide to nuclease degradation. In yet another aspect, the uptake of nanoparticles by a cell is influenced by the density of oligonucleotides or other agents associated with the nanoparticle. As described in WO 08/151,049, incorporated herein by reference in its entirety, a higher density of oligonucleotides or other agents on the surface of a nanoparticle is associated with an increased uptake of nanoparticles by a cell.

A surface density adequate to make the nanoparticles stable and the conditions necessary to obtain it for a desired combination of nanoparticles and agent can be determined empirically. Generally, a surface density of at least 2 pmoles/cm² is adequate to provide nanoparticle-agent compositions having the desired stability and properties (e.g., high cellular uptake and/or sharp melting transitions). In some aspects, the surface density is at least 15 pmoles/cm². Methods are also provided wherein the agent is bound to the nanoparticle at a surface density of at least 2 pmol/cm², at least 3 pmol/cm², at least 4 pmol/cm², at least 5 pmol/cm², at least 6 pmol/cm², at least 7 pmol/cm², at least 8 pmol/cm², at least 9 pmol/cm², at least 10 pmol/cm², at least about 15 pmol/cm², at least about 20 pmol/cm², at least about 25 pmol/cm², at least about 30 pmol/cm², at least about 35 pmol/cm², at least about 40 pmol/cm², at least about 45 pmol/cm², at least about 50 pmol/cm², at least about 55 pmol/cm², at least about 60 pmol/cm², at least about 65 pmol/cm², at least about 70 pmol/cm², at least about 75 pmol/cm², at least about 80 pmol/cm², at least about 85 pmol/cm², at least about 90 pmol/cm², at least about 95 pmol/cm², at least about 100 pmol/cm², at least about 125 pmol/cm², at least about 150 pmol/cm², at least about 175 pmol/cm², at least about 200 pmol/cm², at least about 250 pmol/cm², at least about 300 pmol/cm², at least about 350 pmol/cm², at least about 400 pmol/cm², at least about 450 pmol/cm², at least about 500 pmol/cm², at least about 550 pmol/cm², at least about 600 pmol/cm², at least about 650 pmol/cm², at least about 700 pmol/cm², at least about 750 pmol/cm², at least about 800 pmol/cm², at least about 850 pmol/cm², at least about 900 pmol/cm², at least about 950 pmol/cm², at least about 1000 pmol/cm² or more.

Thus, disclosed herein are modified nanoparticles having an agent attached to at least a portion of the surface at a density of at least 2 pmol/cm², wherein the attachment is through a triazolyl group. The nanoparticle, prior to modification with the agent, has azide groups on its surface, and the agent comprises an alkyne. The mixture of the agent and nanoparticle in the presence of a copper (I) salt and copper ligand allows formation of the triazole group between the alkyne of the agent and the azide of the nanoparticle, as shown in Scheme 1, below.

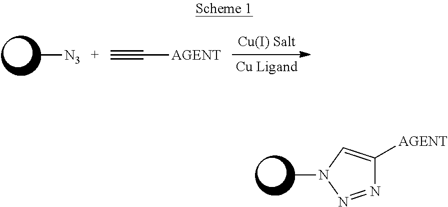

The copper (I) salt can be generated in situ by admixing a copper (II) salt and a reducing agent. The copper (I) or copper (II) salt can comprise any anion compatible with the agent and nanoparticle. Contemplated salts of copper (II) or (I) include, but are not limited to, sulfate, chloride, fluoride, bromide, iodide, phosphate, carbonate, and acetate.

The reducing agent, if present, can be, for example, ascorbic acid, an ascorbate salt (e.g., sodium ascorbate), tris(2-carboxyethyl)phosphine (TCEP), sodium borohydride, 2-mercaptoethanol, dithiothreitol (DTT), hydrazine, lithium aluminum hydride, diisobutylaluminum hydride, oxalic acid, Lindlar catalyst, sulfite compounds, stannous compounds, ferrous compounds, sodium amalgam, and the like. In some specific cases, the reducing agent is sodium ascorbate, TCEP, or a combination thereof.

The copper ligand can comprise a poly(triazole) compound, such as a tris-triazoyl ligand. The presence of the copper ligand protects the agent, such as a biomolecule or oligonucleotide, from degradation by the copper.

The method for preparing the modified nanoparticles illustrated herein is a reaction of an azide on the nanoparticle surface with an alkyne on the agent. However, the reverse reaction to couple the components is also contemplated, i.e., the nanoparticle surface comprises an alkyne and the agent comprises an azide.

In some cases, the modified nanoparticles disclosed herein are prepared via a two phase reaction. Nanoparticles, such as iron oxide nanoparticles, prior to modification with an agent, such as oligonucleotides, are soluble in organic solvents but only sparingly soluble or insoluble in aqueous solutions. In some cases, however, the modified nanoparticle, such as an oligonucleotide-modified nanoparticle, is soluble in aqueous solutions. Therefore, the modified nanoparticle is prepared, in one aspect, using a two-phase system, where the nanoparticle and click chemistry reagents are in the organic phase, and the modified nanoparticle is in the aqueous phase.

Contemplated organic solvents for preparing the modified nanoparticles include, but are not limited to toluene, hexanes, dichloromethane, chloroform, and mixtures thereof. The aqueous phase comprises in certain aspects water, and in some aspects, further comprise salts, buffers, surfactants, and the like. Contemplated salts include without limitation sodium chloride, magnesium chloride, and the like.

### Nanoparticles

Nanoparticles are thus provided which are functionalized to have an azide group on at least a portion of their surface. The size, shape and chemical composition of the nanoparticles contribute to the properties of the resulting azide nanoparticle, and ultimate triazole-modified nanoparticle. These properties include for example, optical properties, optoelectronic properties, electrochemical properties, electronic properties, stability in various solutions, magnetic properties, and pore and channel size variation. Mixtures of nanoparticles having different sizes, shapes and/or chemical compositions, as well as the use of nanoparticles having uniform sizes, shapes and chemical composition, and therefore a mixture of properties are contemplated. Examples of suitable particles include, without limitation, aggregate particles, isotropic (such as spherical particles), anisotropic particles (such as non-spherical rods, tetrahedral, and/or prisms) and core-shell particles, such as those described in U.S. Pat. Nos. 7,238,472 and 7,147,687, the disclosures of which are incorporated by reference in their entirety.

In one embodiment, the nanoparticle is metallic, and in various aspects, the nanoparticle is a colloidal metal. Thus, in various embodiments, nanoparticles of the invention include metal (including for example and without limitation, silver, gold, platinum, aluminum, iron, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. In some embodiments, the nanoparticle comprises superparamagnetic iron oxide nanoparticles (SPIONs). SPIONs have been used in catalysis, biomedicine (27), magnetic resonance imaging, assembly (28), and environmental remediation (29,30).

Also, as described in U.S. Patent Publication No. 2003/0147966, incorporated herein by reference in its entirety, nanoparticles of the invention include those that are available commercially, as well as those that are synthesized, e.g., produced from progressive nucleation in solution (e.g., by colloid reaction) or by various physical and chemical vapor deposition processes, such as sputter deposition. See, e.g., Hayashi, Vac. Sci. Technol. A5(4) δ 1375-84 (1987); Hayashi, Physics Today, 44-60 (1987); MRS Bulletin, January 1990, 16-47.

Nanoparticles can range in size from about 1 nm to about 1000 nm in mean diameter, about 1 nm to about 750 nm in mean diameter, about 1 nm to about 500 nm in mean diameter, about 1 nm to about 400 nm in mean diameter, about 1 nm to about 350 nm in mean diameter, about 1 nm to about 300 nm in mean diameter, about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter. In other aspects, the size of the nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the nanoparticles is from about 30 to about 100 nm, or from about 40 to about 80 nm. The size of the nanoparticles used in a method varies as required by their particular use or application. The variation of size is advantageously used to optimize desired physical characteristics of the nanoparticles, for example, optical properties or the amount of surface area that can be derivatized as described herein.

The process for modifying the surface of the nanoparticle with an azide group depends upon the identity of the nanoparticle. For example, a nanoparticle having an amide, hydroxyl, or other nucleophilic group can react with an N₃—C₁-C₂₀alkylene carboxylic acid, or activated ester thereof, to provide an azide group on at least a potion of the nanoparticle surface, as shown in Scheme 2, below. This reaction can optionally proceed in the presence of a catalyst or coupling reagent, such as include carbodiimides (e.g., DIC or DCC), 1-hydroxybenzotriazole (HOBt), 1-hydroxy-7-azabenzotriazole (HOAt), 2-(1H-benzotriazol-1-yl)-1,1,3,3-tetramethyluronium hexafluorophosphate (HBTU), 2-(7-Aza-1H-benzotriazole-1-yl)-1,1,3,3-tetramethyluronium hexafluorophosphate (HATU), benzotriazol-1-yl-oxytripyrrolidinophosphonium hexafluorophosphate (PyBOP), and the like.

In cases where the nanoparticle surface comprises the alkyne, a similar means for introducing the alkyne moiety can be employed. The HC≡C—C₁-C₂₀alkylene carboxylic acid can be reacted with nucleophile on the nanoparticle surface. It will be appreciated that while a C₁-C₂₀alkylene carboxylic acid is used as the specific example for introducing an alkyne or azide group to the nanoparticle surface, other linking moieties can be employed.

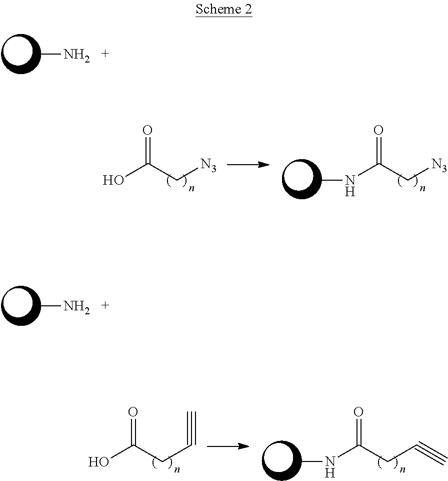

As used herein, the term “alkyl” refers to straight chained and branched hydrocarbon groups, nonlimiting examples of which include methyl, ethyl, and straight chain and branched propyl and butyl groups. The term “alkyl” includes “bridged alkyl,” i.e., a bicyclic or polycyclic hydrocarbon group, for example, norbornyl, adamantyl, bicycle[2.2.2]octyl, bicyclo[2.2.1]heptyl, bicyclo[3.2.1]octyl, or decahydronaphthyl. Alkyl groups optionally can be substituted, for example, with hydroxy (OH), halo, aryl, heteroaryl, ester, carboxylic acid, amido, guanidine, and amino. The term “alkylene” refers to an alkyl group that is substituted. For example, an alkylenehydroxy group is a alkyle group having a hydroxy group somewhere on the alkyl.

As used herein, the term “aryl” refers to a monocyclic or polycyclic aromatic group, preferably a monocyclic or bicyclic aromatic group, e.g., phenyl or naphthyl. Unless otherwise indicated, an aryl group can be unsubstituted or substituted with one or more, and in particular one to four groups independently selected from, for example, halo, alkyl, alkenyl, OCF₃, NO₂, CN, NC, OH, alkoxy, amino, CO₂H, CO₂alkyl, aryl, and heteroaryl. Exemplary aryl groups include, but are not limited to, phenyl, naphthyl, tetrahydronaphthyl, chlorophenyl, methylphenyl, methoxyphenyl, trifluoromethylphenyl, nitrophenyl, 2,4-methoxychlorophenyl, and the like.

As used herein, the term “heteroaryl” refers to a monocyclic or bicyclic ring system containing one or two aromatic rings and containing at least one nitrogen, oxygen, or sulfur atom in an aromatic ring. Unless otherwise indicated, a heteroaryl group can be unsubstituted or substituted with one or more, and in particular one to four, substituents selected from, for example, halo, alkyl, alkenyl, OCF₃, NO₂, CN, NC, OH, alkoxy, amino, CO₂H, CO₂alkyl, aryl, and heteroaryl. Examples of heteroaryl groups include, but are not limited to, thienyl, furyl, pyridyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl.

As used herein, the term “activated ester” refers to a carboxylic acid modified with an activated leaving group enabling reaction with nucleophile (e.g., an amine, a hydroxyl, a thiol) to form a bond and release the leaving group. Activated esters include para-nitrophenyl, hydroxybenzotriazide, and N-hydroxysuccinimide.

### Therapeutic Agents

The nanoparticles can be modified with a therapeutic agent. The therapeutic agent can be an oligonucleotide as described in detail elsewhere herein or a protein, peptide, peptide mimetic, or non-peptide drug. The therapeutic agent can be covalently attached to the nanoparticle, either directly or through a space or linker moiety. In cases where the therapeutic agent is an oligonucleotide, the therapeutic agent can be hybridized to a first or second oligonucleotide of the nanoparticle or attached to the nanoparticle directly or through a spacer or linker moiety.

The therapeutic agent can be selected based on their binding specificity to a ligand expressed in or on a target cell type or a target organ. Alternatively, moieties of this type include a receptor for a ligand on a target cell (instead of the ligand itself), and in still other aspects, both a receptor and its ligand are contemplated in those instances wherein a target cell expresses both the receptor and the ligand. In other aspects, members from this group are selected based on their biological activity, including for example enzymatic activity, agonist properties, antagonist properties, multimerization capacity (including homo-multimers and hetero-multimers). With regard to proteins, therapeutic agents contemplated include full length protein and fragments thereof which retain the desired property of the full length proteins. Fusion proteins, including fusion proteins wherein one fusion component is a fragment or a mimetic, are also contemplated. This group also includes antibodies along with fragments and derivatives thereof, including but not limited to Fab′ fragments, F(ab)₂ fragments, Fv fragments, Fc fragments, one or more complementarity determining regions (CDR) fragments, individual heavy chains, individual light chain, dimeric heavy and light chains (as opposed to heterotetrameric heavy and light chains found in an intact antibody, single chain antibodies (scAb), humanized antibodies (as well as antibodies modified in the manner of humanized antibodies but with the resulting antibody more closely resembling an antibody in a non-human species), chelating recombinant antibodies (CRABs), bispecific antibodies and multispecific antibodies, and other antibody derivative or fragments known in the art.

Non-peptide drugs include compounds that provide a therapeutic benefit, but are not peptides (e.g., are not repeating units of amino acids). Non-peptide drugs can include some peptide-like features, such as, for example, vancomycin, which contains some peptide (e.g., amide) bonds.

### Diagnostic Agents

Diagnostic agents contemplated include radionucleotides, paramagnetic ions, and X-ray imaging agents. Contemplated paramagnetic metal ions include chromium(III), gadolinium(III), iron(II), iron(III), holmium(III), erbium(III), manganese(II), nickel(II), copper(II), neodymium(III), yttrium(III), samarium(III), and dysprosium(III). Contemplated radionuclei include ³H, ¹¹C, ¹⁴C, ¹⁵O, ¹³N, ³²P, ³³P, ³⁵S, ¹⁸F, ¹²⁵I, ¹²⁷I, ¹¹¹In, ¹⁰⁵Rh, ¹⁵³Sm, ⁶⁴Cu, ⁶⁷Cu, ⁶⁷Ga, ¹⁶⁶Ho, ¹⁷⁷Lu, ¹⁸⁶Re, ¹⁸⁸Re, ⁹⁹mTc, ⁸⁶Y and ⁹⁰Y. X-ray imaging agents are selected from the group consisting of gold(III), lead(II), lanthanum(III) and bismuth(III). The diagnostic agent can be attached to the nanoparticle directly or indirectly through a spacer, or the diagnostic agent can be attached to a second agent which is attached to the nanoparticle.

### Fluorophores

In some embodiments, the nanoparticle also comprises a fluorophore. The fluorophore can be covalently attached to the agent or can be itself attached to the nanoparticle surface (directly or indirectly through a spacer). Spacers are described in further detail below.

Non-limiting examples of fluorophores include 5(6)-carboxyfluorescein, 2′,4′,1,4,-tetrachlorofluorescein; 2′,4′,5′,7′,1,4-hexachlorofluorescein, other fluorescein dyes (such as those disclosed in U.S. Pat. Nos. 5,188,934; 6,008,379; 6,020,481, incorporated herein by reference), 6-((7-amino-4-methylcoumarin-3-acetyl)amino)hexanoic acid (Cou), 5(and 6)-carboxy-X-rhodamine (Rox), other rhodamine dyes (such as those disclosed in U.S. Pat. Nos. 5,366,860; 5,847,162; 5,936,087; 6,051,719; 6,191,278; 6,248,884, incorporated herein by reference), benzophenoxazines (such as those disclosed in U.S. Pat. No. 6,140,500, incorporated herein by reference), Cyanine 2 (Cy2) Dye, Cyanine 3 (Cy3) Dye, Cyanine 3.5 (Cy3.5) Dye, Cyanine 5 (Cy5) Dye, Cyanine 5.5 (Cy5.5) Dye, Cyanine 7 (Cy7) Dye, Cyanine 9 (Cy9) Dye, other cyanine dyes (such as disclosed in International Publication No. WO 97/45539), 6-carboxy-4′,5′-dichloro-2′,7′-dimethoxyfluorescein (JOE), 5(6)-carboxy-tetramethyl rhodamine (Tamara), or any one of the Alexa dye series, available from Molecular Probes, Eugene, Oreg.

### Oligonucleotides

As used herein, the term “oligonucleotide” refers to a single-stranded oligonucleotide having natural and/or unnatural nucleotides. Throughout this disclosure, nucleotides are alternatively referred to as nucleobases. The oligonucleotide can be a DNA oligonucleotide, an RNA oligonucleotide, or a modified form of either a DNA oligonucleotide or an RNA oligonucleotide.

Naturally occurring nucleobases include adenine (A), guanine (G), cytosine (C), thymine (T), and uracil (U), as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopurine, 5-methylcytosine (mC), 5-(C₃-C₆)-alkynyl-cytosine, 5-fluorouracil, 5-bromouracil, pseudoisocytosine, 2-hydroxy-5-methyl-4-triazolopyridin, isocytosine, isoguanine, inosine, and the “unnatural” nucleobases include those described in U.S. Pat. No. 5,432,272 and Freier et al. Nucleic Acids Research, 25:4429-4443 (1997), each of which is incorporated by reference in its entirety. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808; in Sanghvi, Antisense Research and Application, Crooke and B. Lebleu, eds., CRC Press, 1993, Chapter 15; in Englisch et al., Angewandte Chemie, International Edition, 30:613-722 (1991); and in the Concise Encyclopedia of Polymer Science and Engineering, J. I. Kroschwitz Ed., John Wiley & Sons, 1990, pages 858-859, Cook, Anti-Cancer Drug Design, 6, 585-607 (1991), each of which is hereby incorporated by reference in its entirety. Nucleobase also includes compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include pyrrole, diazole, and triazole derivatives, including those universal bases known in the art. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. The oligonucleotide incorporated with the universal base analogues is able to function as a probe in hybridization, and as a primer in PCR and DNA sequencing. Examples of universal bases include but are not limited to 5′-nitroindole-2′-deoxyriboside, 3-nitropyrrole, inosine and pypoxanthine.

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of each are incorporated herein by reference in their entireties.

Modified oligonucleotides includes oligonucleotides wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of each are each incorporated by reference herein.

Other linkages between nucleotides and unnatural nucleotides contemplated for the disclosed oligonucleotides include those described in U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920; U.S. Patent Publication No. 20040219565; International Patent Publication Nos. WO 98/39352 and WO 99/14226; Mesmaeker et al., Current Opinion in Structural Biology 5:343-355 (1995) and Susan M. Freier and Karl-Heinz Altmann, Nucleic Acids Research, 25:4429-4443 (1997), the disclosures of each are incorporated by reference in their entirety.

Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically. Non-naturally occurring nucleobases can be incorporated into the oligonucleotide, as well. See, e.g., Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002), the disclosures of which are incorporated by reference in their entirety.

The oligonucleotide can be bound to the nanoparticle through a 5′ linkage and/or the oligonucleotide is bound to the nanoparticle through a 3′ linkage. In various aspects, at least one oligonucleotide is bound through a spacer to the nanoparticle. In these aspects, the spacer is an organic moiety, a polymer, a water-soluble polymer, a nucleic acid, a polypeptide, and/or an oligosaccharide. Methods of functionalizing the oligonucleotides to attach to a surface of a nanoparticle are well known in the art. See Whitesides, Proceedings of the Robert A. Welch Foundation 39th Conference On Chemical Research Nanophase Chemistry, Houston, Tex., pages 109-121 (1995), or Mucic et al. Chem. Comm. 555-557 (1996), the disclosures of which are incorporated by reference in their entirety. The oligonucleotide can be modified to include an alkyne moiety at a terminus, as described in the below examples, or other synthetic means available to the skilled artisan.

Nanoparticles disclosed herein can be functionalized with an oligonucleotide, or modified form thereof, which is from about 15 to about 200 nucleotides in length. Also contemplated are oligonucleotides of about 15 to about 150 nucleotides in length, about 15 to about 100 nucleotides in length, about 15 to about 80 nucleotides in length, about 15 to about 60 nucleotides in length, about 15 to about 50 nucleotides in length about 15 to about 45 nucleotides in length, about 15 to about 40 nucleotides in length, about 15 to about 35 nucleotides in length, about 15 to about 30 nucleotides in length, about 15 to about 25 nucleotides in length, about 15 to about 20 nucleotides in length, and all oligonucleotides intermediate in length of the sizes specifically disclosed to the extent that the oligonucleotide is able to achieve the desired result. Accordingly, oligonucleotides of 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, and 200 nucleotides in length are contemplated.

### Oligonucleotide Features

In various aspects, the nanoparticles disclosed herein comprise an oligonucleotide that can modulate expression of a gene product expressed from a target polynucleotide. Accordingly, antisense oligonucleotides which hybridize to a target polynucleotide and inhibit translation, siRNA oligonucleotides which hybridize to a target polynucleotide and initiate an RNAse activity (for example RNAse H), triple helix forming oligonucleotides which hybridize to double-stranded polynucleotides and inhibit transcription, and ribozymes which hybridize to a target polynucleotide and inhibit translation, are contemplated.

In various aspects, a plurality of oligonucleotides can be attached to the nanoparticle. As a result, each oligonucleotide-modified nanoparticle can have the ability to bind to a plurality of target compounds. In various aspects, the plurality of oligonucleotides can be identical. It is also contemplated wherein the plurality of oligonucleotides includes about 10 to about 100,000 oligonucleotides, about 10 to about 90,000 oligonucleotides, about 10 to about 80,000 oligonucleotides, about 10 to about 70,000 oligonucleotides, about 10 to about 60,000 oligonucleotides, 10 to about 50,000 oligonucleotides, 10 to about 40,000 oligonucleotides, about 10 to about 30,000 oligonucleotides, about 10 to about 20,000 oligonucleotides, about 10 to about 10,000 oligonucleotides, and all numbers of oligonucleotides intermediate to those specifically disclosed to the extent that the oligonucleotide-modified nanoparticle is able to achieve the desired result.

Thus, each nanoparticle provided herein can have a plurality of oligonucleotides attached to it. As a result, each modified nanoparticle has the ability to bind to a plurality of oligonucleotides and/or target polynucleotides having a sufficiently complementary sequence. For example, if a specific mRNA is targeted, a single nanoparticle has the ability to bind to multiple copies of the same transcript. In one aspect, methods are provided wherein the nanoparticle is functionalized with identical oligonucleotides, i.e., each oligonucleotide has the same length and the same sequence. In other aspects, the nanoparticle is functionalized with two or more oligonucleotides which are not identical, i.e., at least one of the attached oligonucleotides differ from at least one other attached oligonucleotide in that it has a different length and/or a different sequence. In aspects wherein different oligonucleotides are associated with the nanoparticles, these different oligonucleotides bind to the same single target polynucleotide but at different locations, or bind to different target polynucleotides which encode different gene products. Accordingly, in various aspects, a single modified nanoparticle may be used in a method to inhibit expression of more than one gene product. Oligonucleotides are thus used to target specific polynucleotides, whether at one or more specific regions in the target polynucleotide, or over the entire length of the target polynucleotide as the need may be to effect a desired level of inhibition of gene expression.

Accordingly, in some aspects, the oligonucleotides are designed with knowledge of the target sequence. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

Alternatively, oligonucleotides are selected from a library. Preparation of libraries of this type is well know in the art. See, for example, U.S. Patent Publication No. 2005/0214782, incorporated by reference herein. Preparation of siRNA oligonucleotide libraries is generally described in U.S. Patent Publication No. 2005/0197315, the disclosure of which is incorporated herein by reference in its entirety.

Further provided are embodiments wherein the oligonucleotide is functionalized in such a way that the oligonucleotide is released from the nanoparticle after the nanoparticle enters a cell. In general, an oligonucleotide can be release from the surface of a nanoparticle using either chemical methods, photon release, and changes in ionic or acid/base environment. In some cases, the oligonucleotide is attached to the nanoparticle through a spacer capable of releasing the oligonucleotide, e.g., contains a releasable linker moiety, such as an acid or base labile moiety, a photo-labile moiety. Spacers are described in greater detail below.

In one aspect of this embodiment, the oligonucleotide is attached to the nanoparticle via an acid-labile moiety and once the modified nanoparticle is taken into the cell via, for example, an endosome, acidification of the endosome (a normal part of endosomal uptake) releases the oligonucleotides. This aspect is particular useful in instances where the intent is to saturate the cell with for example, an siRNA. Release from the nanoparticle would improve kinetics and resolve potential steric hindrance problems in embodiments where siRNA. RNAi for modulating gene expression is well known in the art and generally described in, for example, U.S. Patent Publication No. 2006/0019917, U.S. Patent Publication No. 2006/0008907 and U.S. Patent Publication No. 2005/0059016, the disclosures of which are incorporated herein by reference in their entireties.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of nanostructure disclosed herein. In other words, methods provided embrace those which results in essentially any degree of inhibition of expression of a target gene product.

The degree of inhibition is determined in vivo from a body fluid sample or from a biopsy sample or by imaging techniques well known in the art. Alternatively, the degree of inhibition is determined in a cell culture assay, generally as a predictable measure of a degree of inhibition that can be expected in vivo resulting from use of a specific type of nanoparticle and a specific oligonucleotide.

### Oligonucleotide Sequences and Hybridization

Each oligonucleotide-modified nanoparticle has the ability to hybridize to a portion of a second oligonucleotide having a sequence sufficiently complementary. In some cases, the second oligonucleotide is a target oligonucleotide (e.g., a portion of a polynucleotide that encode a gene product). In various aspects, the oligonucleotides of oligonucleotide-modified nanoparticle are 100% complementary to a portion of the second oligonucleotide, i.e., a perfect match, while in other aspects, the oligonucleotides are at least (meaning greater than or equal to) about 95% complementary to portions of the second oligonucleotide over the length of the oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to portions of the second oligonucleotide over the length of the oligonucleotide(s).

“Hybridization” means an interaction between two strands of nucleic acids by hydrogen bonds in accordance with the rules of Watson-Crick DNA complementarity, Hoogstein binding, or other sequence-specific binding known in the art. Hybridization can be performed under different stringency conditions known in the art. These hybridization conditions are well known in the art and can readily be optimized for the particular system employed. See, e.g., Sambrook et al., Molecular Cloning: A Laboratory Manual (2nd ed. 1989). Preferably stringent hybridization conditions are employed. Under appropriate stringency conditions, hybridization between the two complementary strands can reach about 60% or above, about 70% or above, about 80% or above, about 90% or above, about 95% or above, about 96% or above, about 97% or above, about 98% or above, or about 99% or above.

### Spacers

In certain aspects, modified nanoparticles are contemplated which include those wherein an agent is attached to the nanoparticle through a spacer. “Spacer” as used herein means a moiety that does not participate in the diagnostic or therapeutic properties of the agent but which serves to increase distance between the nanoparticle and the agent, or to increase distance between individual agents when attached to the nanoparticle in multiplicity (e.g., more than one copy of the agent and/or two or more agents). Thus, spacers are contemplated being located between individual agents in tandem, whether the agents are the same or different. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, a polyethylene glycol, or combinations thereof.

### Surface Density

Density of oligonucleotides on the surface of a nanoparticle has been shown to modulate specific polypeptide interactions with the oligonucleotide on the surface and/or with the nanoparticle itself. Under various conditions, some polypeptides may be prohibited from interacting with oligonucleotides associated with a nanoparticle based on steric hindrance caused by the density of oligonucleotides. In aspects where interaction of oligonucleotides with polypeptides that are otherwise precludes by steric hindrance is desirable, the density of oligonucleotides on the nanoparticle surface is decreased to allow the polypeptide to interact with the oligonucleotide.

Oligonucleotide surface density has also been shown to modulate stability of the polynucleotide associated with the nanoparticle. In one embodiment, an RNA oligonucleotide associated with a nanoparticle is provided wherein the RNA oligonucleotide has a half-life that is at least substantially the same as the half-life of an identical RNA oligonucleotide that is not associated with a nanoparticle. In other embodiments, the RNA oligonucleotide associated with the nanoparticle has a half-life that is about 5% greater, about 10% greater, about 20% greater, about 30% greater, about 40% greater, about 50% greater, about 60% greater, about 70% greater, about 80% greater, about 90% greater, about 2-fold greater, about 3-fold greater, about 4-fold greater, about 5-fold greater, about 6-fold greater, about 7-fold greater, about 8-fold greater, about 9-fold greater, about 10-fold greater, about 20-fold greater, about 30-fold greater, about 40-fold greater, about 50-fold greater, about 60-fold greater, about 70-fold greater, about 80-fold greater, about 90-fold greater, about 100-fold greater, about 200-fold greater, about 300-fold greater, about 400-fold greater, about 500-fold greater, about 600-fold greater, about 700-fold greater, about 800-fold greater, about 900-fold greater, about 1000-fold greater, about 5000-fold greater, about 10,000-fold greater, about 50,000-fold greater, about 100,000-fold greater, about 200,000-fold greater, about 300,000-fold greater, about 400,000-fold greater, about 500,000-fold greater, about 600,000-fold greater, about 700,000-fold greater, about 800,000-fold greater, about 900,000-fold greater, about 1,000,000-fold greater or more than the half-life of an identical RNA oligonucleotide that is not associated with a nanoparticle.

### Target Polynucleotides

In various aspects, the disclosed nanoparticles are modified with an oligonucleotide that is a target for an intracellular polynucleotide or are co-administered with an oligonucleotide that is a target for an intracellular polynucleotide. The target polynucleotide can be eukaryotic, prokaryotic, viral, or fungal.

In various embodiments, methods provided include those wherein the target polynucleotide is a mRNA encoding a gene product and translation of the gene product is inhibited, or the target polynucleotide is DNA in a gene encoding a gene product and transcription of the gene product is inhibited. In methods wherein the target polynucleotide is DNA, the polynucleotide is in certain aspects DNA which encodes the gene product being inhibited. In other methods, the DNA is complementary to a coding region for the gene product. In still other aspects, the DNA encodes a regulatory element necessary for expression of the gene product. “Regulatory elements” include, but are not limited to enhancers, promoters, silencers, polyadenylation signals, regulatory protein binding elements, regulatory introns, ribosome entry sites, and the like. In still another aspect, the target polynucleotide is a sequence which is required for endogenous replication.

The terms “start codon region” and “translation initiation codon region” refer to a portion of an mRNA or gene that encompasses contiguous nucleotides in either direction (i.e., 5′ or 3′) from a translation initiation codon. Similarly, the terms “stop codon region” and “translation termination codon region” refer to a portion of such an mRNA or gene that encompasses contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the oligonucleotides on the functionalized nanoparticles.

Other target regions include the 5′ untranslated region (5′UTR), the portion of an mRNA in the 5′ direction from the translation initiation codon, including nucleotides between the 5′ cap site and the translation initiation codon of an mRNA (or corresponding nucleotides on the gene), and the 3′ untranslated region (3′UTR), the portion of an mRNA in the 3′ direction from the translation termination codon, including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site.

For prokaryotic target polynucleotides, in various aspects, the polynucleotide is genomic DNA or RNA transcribed from genomic DNA. For eukaryotic target polynucleotides, the polynucleotide is an animal polynucleotide, a plant polynucleotide, a fungal polynucleotide, including yeast polynucleotides. As above, the target polynucleotide is either a genomic DNA or RNA transcribed from a genomic DNA sequence. In certain aspects, the target polynucleotide is a mitochondrial polynucleotide. For viral target polynucleotides, the polynucleotide is viral genomic RNA, viral genomic DNA, or RNA transcribed from viral genomic DNA.

Methods for inhibiting gene product expression provided include those wherein expression of the target gene product is inhibited by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or 100% compared to gene product expression in the absence of an oligonucleotide-functionalized nanoparticle. In other words, methods provided embrace those which results in essentially any degree of inhibition of expression of a target gene product.

### Cancer

The disclosed modified nanoparticles can be used to deliver a therapeutic, such as a chemotherapeutic, to a cell (e.g., a cancerous cell), and thus, are useful for treating a wide variety of diseases, such as cancers, including carcinomas, sarcomas, leukemias, and lymphomas. The modified nanoparticle can comprise a targeting agent, such as an antibody which recognizes a specific cancer cell, to direct the modified nanoparticle to specific (e.g., cancerous) cells and deliver the (therapeutic) agent to the specific cell.

Chemotherapeutic agents that can be used include, but are not limited to, alkylating agents, antimetabolites, hormones and antagonists thereof, radioisotopes, antibodies, as well as natural products, and combinations thereof. For example, an inhibitor compound of the present invention can be administered with antibiotics, such as doxorubicin and other anthracycline analogs, nitrogen mustards, such as cyclophosphamide, pyrimidine analogs such as 5-fluorouracil, cis-platin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of an inhibitor compound with another treatment modality, e.g., surgery or radiation. Additional chemotherapeutic agents useful in the invention include hormones and antagonists thereof, radioisotopes, antibodies, non-peptide drugs, and combinations thereof. Further examples of chemotherapeutic agents include, for example, camptothecin, carboplatin, cisplatin, daunorubicin, doxorubicin, interferon (α, β, γ), irinotecan, hydroxyurea, chlorambucil, 5-fluorouracil (5-FU), methotrexate, 2-chloroadenosine, fludarabine, azacytidine, gemcitabine, pemetrexed, interleukin 2, irinotecan, docetaxel, paclitaxel, topotecan, and therapeutically effective analogs and derivatives of the same. More examples of chemotherapeutic agents useful for the method of the present invention are listed in the following table.

TABLE

| | | |
|---|---|---|
| Alkylating agents | Nitrogen mustards | mechlorethamine |
| cyclophosphamide | ifosfamide | melphalan |
| chlorambucil | Nitrosoureas | carmustine (BCNU) |
| lomustine (CCNU) | semustine (methyl- | Ethylenimine/Methylmelamine |
| | CCNU) | |
| thriethylenemelamine | triethylene | (thiotepa) |
| (TEM) | thiophosphoramide | |
| hexamethylmelamine | (HMM, altretamine) | Alkyl sulfonates |
| busulfan | Triazines | dacarbazine (DTIC) |
| Antimetabolites | Folic Acid analogs | methotrexate |
| trimetrexate | Pyrimidine analogs | 5-fluorouracil |
| fluorodeoxyuridine | gemcitabine | cytosine arabinoside |
| (AraC, cytarabine) | 5-azacytidine | 2,2′-difluorodeoxycytidine |
| Purine analogs | 6-mercaptopurine | 6-thioguanine |
| azathioprine | 2′-deoxycoformycin | (pentostatin) |
| erythrohydroxynonyladenine | fludarabine phosphate | 2-chlorodeoxyadenosine |
| (EHNA) | | |
| (cladribine, 2-CdA) | multitargeted antifolate | Type I Topoisomerase |
| | | Inhibitors |
| camptothecin | topotecan | irinotecan |
| Natural products | Antimitotic drugs | paclitaxel |
| Vinca alkaloids | vinblastine (VLB) | vincristine |
| vinorelbine | Taxotere ® (docetaxel) | estramustine |
| estramustine phosphate | etoposide | teniposide |
| Epipodophylotoxins | | |
| Antibiotics | actimomycin D | daunomycin (rubidomycin) |
| doxorubicin (adriamycin) | mitoxantroneidarubicin | bleomycinsplicamycin |
| | | (mithramycin) |
| mitomycinC | dactinomycin | Enzymes |
| L-asparaginase | Biological response | interferon-alpha |
| | modifiers | |
| IL-2 | G-CSF | GM-CSF |
| Differentiation Agents | retinoic acid | Radiosensitizers |
| | derivatives | |
| metronidazole | misonidazole | desmethylmisonidazole |
| pimonidazole | etanidazole | nimorazole |
| RSU 1069 | EO9 | RB 6145 |
| SR4233 | nicotinamide | 5-bromodeozyuridine |
| 5-iododeoxyuridine | bromodeoxycytidine | Miscellaneous agents |
| Platinium coordination | cis-platin | carboplatin |
| complexes | | |
| oxaliplatin | Anthracenedione | mitoxantrone |
| Substituted urea | hydroxyurea | Methylhydrazine derivatives |
| N-methylhydrazine (MIH) | procarbazine | Adrenocortical suppressant |
| mitotane (o,p′-DDD) | ainoglutethimide | Cytokines |
| interferon (α, β, γ) | interleukin-2 | Adrenocorticosteroids/ |
| | Hormones and | antagonists |
| | antagonists | |
| prednisone and equivalents | dexamethasone | ainoglutethimide |
| Progestins | hydroxyprogesterone | medroxyprogesterone acetate |
| | caproate | |
| megestrol acetate | Estrogens | diethylstilbestrol |
| ethynyl estradiol/ | Antiestrogen | tamoxifen |
| equivalents | | |
| Androgens | testosterone propionate | fluoxymesterone/equivalents |
| Antiandrogens | flutamide | gonadotropin-releasing |
| hormone analogs | leuprolide | Nonsteroidal antiandrogens |
| flutamide | Photosensitizers | hematoporphyrin derivatives |
| Photofrin ® | benzoporphyrin | Npe6 |
| | derivatives | |
| tin etioporphyrin (SnET2) | pheoboride-a | bacteriochlorophyll-a |
| naphthalocyanines | phthalocyanines | zinc phthalocyanines |
| Growth Factor Receptor | EGFR antagonists | HER-2 antagonists |
| Antagonists | | |

Carcinomas that can be treated using a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; non-Hodgkin's lymphoma, and the like.

All patents, publications and references cited herein are hereby fully incorporated by reference. In case of conflict between the present disclosure and incorporated patents, publications and references, the present disclosure should control.

### EXAMPLES

Oligonucleotides were synthesized on an Expedite 8909 Nucleotide Synthesis System (ABI) using standard solid-phase phosphoramidite methodology (Supporting Information). Bases and reagents were purchased from Glen Research. The oligonucleotides were purified using reverse-phase high performance liquid chromatography (RP-HPLC) using a Varian Microsorb C18 column (10 μm, 300×10 mm) with 0.03 M triethylammonium acetate (TEAA), pH 7 and a 1%/min gradient of 100% CH₃CN at a flow rate of 3 mL/min, while monitoring the UV signal of DNA at 254 nm.

To prepare azide functionalized SPIONs, 15 mg of succinimidyl 4-azidobutyrate (Glen Research) dissolved in 100 uL of DMSO were added to 1 mL of 1 mg/mL (1 uM) 10 nm aminated SPIONs (Ocean Nanotech) and reacted in borate buffer (pH 8.5). The solution was incubated overnight at room temperature, followed by centrifugation to isolate the particles. The supernatent was removed, and the particles were redispersed in phosphate buffer (0.01% SDS, 10 mM phosphate, pH 7.4) with sonication. This step was repeated three times to eliminate any residual azidobutyrate NHS ester. These particles were less stable than the amine functionalized particles and so were stored at 4° C. for no more than a week before conjugation to DNA.

In a typical conjugation experiment (Scheme 3), about 20 nmol of an oligonucleotide modified with a terminal alkyne (for example, 1, SEQ ID NO: 1: 5′ alkyne-A₁₀-ATT-ATC-ACT 3′; and 2, SEQ ID NO: 2: 5′ alkyne-A₁₀-AGT-GAT-AAT 3′) were added to a DMSO solvated mixture of a copper (II) salt (e.g., the sulfate salt, CuSO₄), a copper ligand (e.g., THPT (tris-hydroxylpropyl triazolyl)), and a reducing agent (e.g., ascorbic acid (AA)). The THPT ligand prevents possible degradation of oligonucleotides in the presence of copper (I) (21). This mixture was prepared in a 1:2:10 ratio of the respective components, resulting in final concentrations of 200 μM CuSO₄, 400 μM THPT, and 2 mM AA. Then, 100 pmol of azide-functionalized particles were added to this solution. The reaction was halted by centrifugation, and the nanoparticles were isolated and resuspended in 0.15M PBS. This process was repeated three times. The ratio of the catalytic elements to one another are important in producing stable conjugates, because a large excess of ligand increases the stability of Cu(I) ion in solution (31) and a 10 fold excess of AA keeps copper reduced over the course of the reaction.

In an alternative, modified nanoparticles are prepared using a two-phase system synthesis. Azide functionalized nanoparticles, such as SPIONs, are dissolved in an organic solvent, such as toluene, hexanes, dichloromethane, chloroform, or a mixture thereof. An aqueous solution comprising water and optionally a salt and/or buffer is added to the organic solution. A copper salt, oligonucleotides having an alkyne functional group, and optionally, a copper ligand, are added to the resulting two phase system. The resulting oligonucleotide-modified nanoparticles (e.g., oligonucleotide—SPIONs) are pulled into the aqueous phase because the modified nanoparticles are highly stable in the aqueous phase. By addition of the oligonucleotides, the particles migrate through the phase barrier and become more stable and/or more soluble as more oligonucleotides are appended to the surface of the nanoparticle. The modified nanoparticles can be collected from the aqueous phase and the by products of the reaction stay in the organic phase.

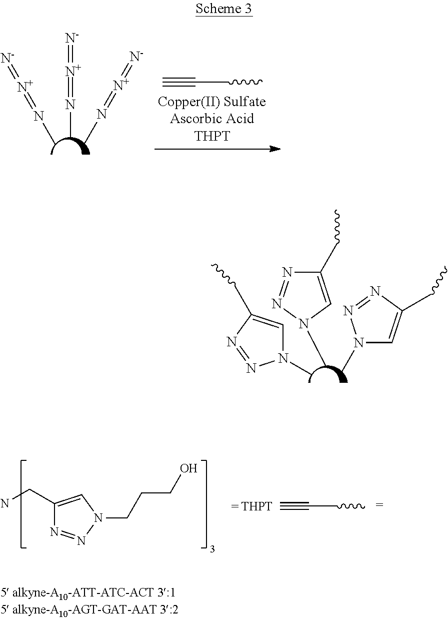

To study the extent of the conjugation reaction and the properties of the resulting oligonucleotide-SPION conjugates, two batches of particles were functionalized with complementary oligonucleotide sequences (SEQ ID NOs: 1 and 2, Scheme 1). Unmodified 10 nm SPIONs are well-dispersed and are too small to be rapidly pulled out of solution with a conventional bar magnet. When the DNA-SPION particles are functionalized with SEQ ID NOs: 1 and 2, they retain their stability and can be suspended in solution without evidence of aggregation. When particles functionalized with SEQ ID NOs: 1 and 2, respectively, are combined in equal amounts, the particles aggregate within a short period of time (e.g., 1-2 hours) and can be manipulated easily by a magnetic field. Because this process is due to DNA hybridization interactions, it is reversible, and upon heating, the aggregates disperse and the particles are released. The broad absorption of iron oxide in the visible region of the spectrum allows one to easily distinguish aggregated particles from suspended ones spectroscopically or by eye, as the aggregated particles form a heterogeneous suspension that does not efficiently absorb light.

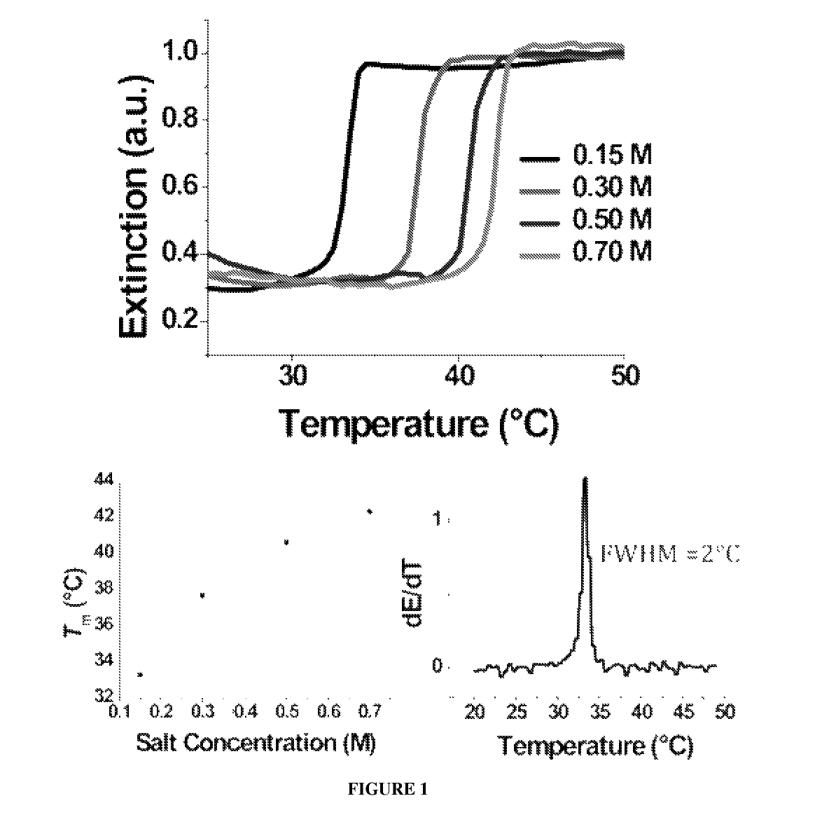

The ability to distinguish aggregated particles from freely suspended particles was used to further analyze the binding properties of the conjugates via oligonucleotide melting experiments. The reversible hybridization process was monitored at 260 nm as a function of temperature (FIG. 1). The oligonucleotide-SPION conjugates exhibit sharp cooperative melting transitions (full width at half maximum of about 2° C.), which are characteristic of particles functionalized with a dense monolayer of oligonucleotides (32). The melting temperature of the aggregates increases as a function of increasing salt concentration (0.15 to 0.7 M), a reflection of increased charge screening of the oligonucleotides involved in hybridization (14).

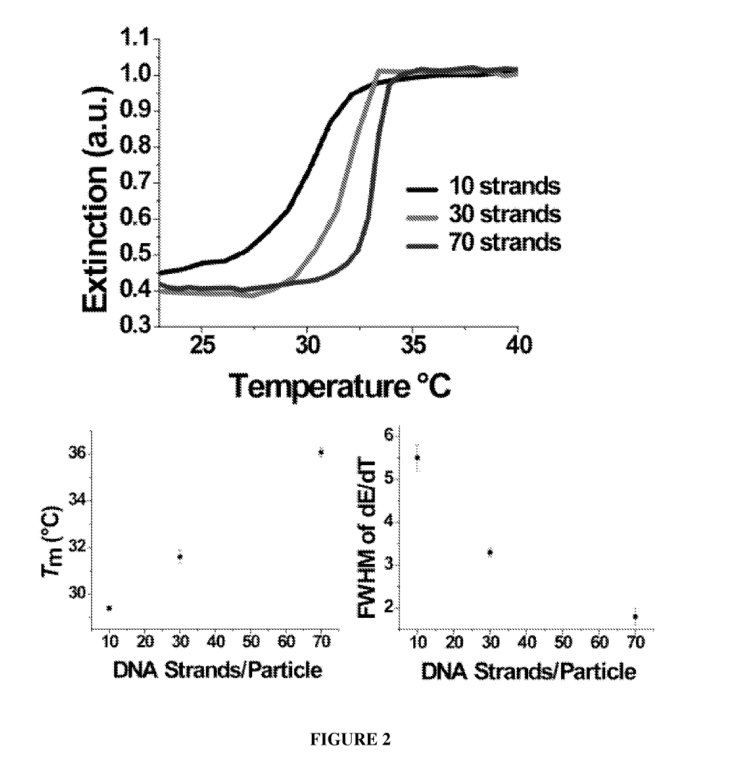

Oligonucleotide loading can be controlled with this system by quenching the Cu(I) reaction at different time points. Particles with densities of 3.18×10¹² to 2.29×10¹³ oligos/cm² (about 10-70 strands per 10 nm particle) have been prepared and their hybridization and subsequent melting properties studied (FIG. 2). In general, higher loading results in a higher Tₙ, and a more narrow transition (32).

Polyvalent oligonucleotide-gold nanoparticle conjugates, despite their high negative surface charge, exhibit cellular uptake as a result of dense oligonucleotide loading (33). The particles are able to attract a layer of signaling proteins, which has been hypothesized to facilitate cellular internalization of the particles (33). Having established that this novel click strategy for preparing SPION particles is effective at creating particles with high surface densities of nucleic acids, we next investigated their ability to enter HeLa cells (human cervical cancer). In a typical experiment, cells were cultured on slide chambers, incubated with 200 μl of a 50 pM nanoparticle solution for 12 hours, and then imaged using confocal microscopy. The resulting oligonucleotide-SPION treated-HeLa cells were highly fluorescent, with fluorescence primarily seen in the cytoplasm, consistent with observations made from oligonucleotide-gold nanoparticle experiments (8). Significantly, these results show that like the analogous oligonucleotide-gold nanoparticle conjugates, the polyvalent oligonucleotide-conjugated SPIONs readily enter cells without the need for transfection agents.

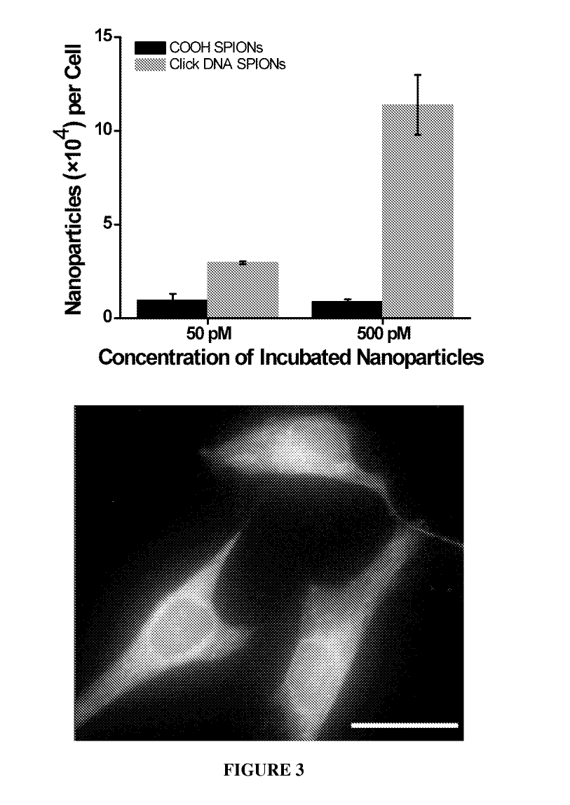

In order to quantify the uptake efficiency of the SPION-oligonucleotide conjugates, the iron content of the cells was examined using inductively coupled plasma mass spectroscopy (ICP-MS). HeLa Cells were cultured in 24-well plates, incubated with 200 μl solutions of 50 pM and 500 pM oligonucleotide-SPIONs for 24 hours, and collected for the iron content. Carboxylic acid modified SPION of the same concentrations were used as a control. Due to the dense functionalization of oligonucleotides on the surface of the nanoparticles, the oligonucleotide-SPIONs enter cells (50,000-150,000 number per cell) while SPIONS in solution exhibit a consistently lower uptake (about 10,000 per cell) when compared to the oligonucleotide-SPIONS (FIG. 3). This is further evidence that a dense layer of oligonucleotides on a nanoparticle surface, regardless of core, can mediate cellular uptake without transfection agents. This is significant because most methods for delivery of genetic material, utilizing SPIONs, require the use of potentially toxic transfection agents (34) or targeting epitopes (35).

HeLa cells (ATCC) were maintained in Eagle's Minimal Essential Medium (EMEM), with 10% heat inactivated fetal bovine serum and maintained at 37° C. in 5% CO2. Cells were grown on Lab-Tek®II Chamber #1.5 German Coverglass System (Nalge Nunc International). For imaging, sterile filtered DNA-SPIONs (particle concentration, 50 pM) were added directly to the cell culture media. After 12 hours of treatment, the cells were washed with PBS and fresh EMEM was added. Live cells were stained with TubulinTracker™ Green reagent, and Hoechst 33342 (Invitrogen).

For inductively coupled plasma mass spectrometry (ICP-MS) analysis experiments, sterile filtered DNA-SPIONs or COOH—SPIONs were added directly to the cell culture media of adherent cells in concentrations of 50 and 500 pM. 24 hours after nanoparticles addition, the cells were washed 3 times in PBS buffer, collected and counted using a Guava EasyCyte mini (Guava Technologies). To prepare samples for ICP-MS (Thermo-Fisher), the cells were dissolved with neat nitric acid at 60° C. overnight. The iron content of the cell digest was determined by ICP-MS. Each cell sample was prepared in a matrix consisting of 2% HNO₃, 2% HCl, 5 ppb Indium (internal standard), and Nanopure™ water. In order to extract the number of nanoparticles taken up by each cell, the number of nanoparticles must be calculated based on the concentration of iron found in the sample. All ICP experiments were preformed in triplicate and values obtained were averaged.

### REFERENCES

(1) Mirkin, et al, Nature 1996, 382, 607.
(2) Alivisatos, et al., Nature 1996, 382, 609.
(3) Long, et al., J. Phys. Chem. B 2006, 110, 2918.
(4) Bao, et al., Nucleic Acids Research 2005, 33.
(5) Graham, et al., Nature Nanotechnology 2008, 3, 548.
(6) Taton, et al., Science 2000, 289, 1757.
(7) Seferos, et al., Nano Lett. 2008.
(8) Rosi, et al., Science 2006, 312, 1027.
(9) Park, et al., Nature 2008, 451, 553.
(10) Nykypanchuk, et al., Nature 2008, 451, 549.
(11) Lee, et al., Anal. Chem. 2008, 80, 6805.
(12) Nam, et al., Science 2003, 301, 1884.
(13) Liu, et al., J. Am. Chem. Soc. 2003, 125, 6642.
(14) Lee, et al., Nano Lett. 2007, 7, 2112.
(15) Medintz, et al., Nano Letters 2007, 7, 1741.
(16) Hilliard, et al., Anal. Chim. Acta 2002, 470, 51.
(17) Derfus, et al., Adv. Mater. 2007, 19, 3932.
(18) Kolb, et al., Angew. Chem. Int. Ed. 2001, 40, 2004.
(19) Wang, et al., J. Am. Chem. Soc. 2003, 125, 3192.
(20) Fleming, et al., Chem. Mater. 2006, 18, 2327.
(21) Kumar, et al., J. Am. Chem. Soc. 2007, 129, 6859.
(22) Rozkiewicz, et al., Chembiochem 2007, 8, 1997.
(23) Gole, et al., Langmuir 2008, 24, 266.
(24) von Maltzahn, et al., Bioconjugate Chem. 2008, 19, 1570.
(25) Fischler, et al., Chem. Commun. 2008, 169.
(26) Gramlich, et al., Angew. Chem. Int. Ed. 2008, 47, 8350.
(27) Sandhu, et al., Bioconjugate Chem. 2002, 13, 3.
(28) Srivastava, et al., J. Am. Chem. Soc. 2007, 129, 11776.
(29) Jun, et al., Acc. Chem. Res. 2008, 41, 179.
(30) Deng, et al., J. Am. Chem. Soc. 2008, 130, 28.
(31) Chan, et al., Organic Letters 2004, 6, 2853.
(32) Jin, et al., J. Am. Chem. Soc. 2003, 125, 1643.
(33) Giljohann, et al., Nano Lett. 2007, 7, 3818.
(34) Plank, et al., Biological Chemistry 2003, 384, 737.
(35) Lee, et al., Angew. Chem. Int. Ed. 2009, 48, 4174.

1. A nanoparticle comprising an agent attached to at least a portion of the nanoparticle surface through a triazolyl group, wherein the agent has a surface density of at least 5 pmol/cm² and the nanoparticle is magnetic or paramagnetic.

2. (canceled)

3. The nanoparticle of claim 1, wherein the nanoparticle comprises iron oxide.

4. The nanoparticle of claim 1, wherein the surface density is at least 10 pmol/cm².

5. The nanoparticle of claim 1, wherein the nanoparticle has a diameter of 2 nm to 1000 nm.

6. (canceled)

7. The nanoparticle of claim 1, wherein the agent comprises an oligonucleotide having 5 to 200 nucleobases.

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The nanoparticle of claim 7, wherein the oligonucleotide is bound to the nanoparticle through a 5′ linkage.

28. The nanoparticle of claim 7, wherein the oligonucleotide is bound to the nanoparticle through a 3′ linkage.

29. The nanoparticle of claim 7, wherein the oligonucleotide is bound to the nanoparticle through a spacer.

30. The nanoparticle of claim 29, wherein the spacer is a polymer.

31. (canceled)

32. (canceled)

33. (canceled)

34. (canceled)

35. The nanoparticle of claim 1, further comprising a second agent.

36. The nanoparticle of claim 35, wherein the second agent comprises an oligonucleotide, a protein, a peptide, an antibody, a non-peptide drug, or combinations thereof.

37. The nanoparticle of claim 36, where the second agent comprises a second oligonucleotide having 5 to 200 nucleobases.

38. (canceled)

39. (canceled)

40. (canceled)

41. (canceled)

42. (canceled)

43. (canceled)

44. (canceled)

45. (canceled)

46. (canceled)

47. A method of preparing a nanoparticle of claim 1, comprising admixing a nanoparticle having an azide group on at least a portion of the nanoparticle surface, an agent having an alkyne group, a copper (II) salt, a reducing agent, and a copper ligand to form the nanoparticle modified with the agent on the nanoparticle surface through a triazolyl group.

48. The method of claim 47, wherein the reducing agent comprises ascorbic acid or a salt thereof.

49. The method of claim 47, wherein the copper ligand comprises a tris-triazole group.

50. The method of claim 47, wherein the agent is an oligonucleotide, a protein, a peptide, an antibody, a non-peptide drug, or combinations thereof.

51. (canceled)

52. (canceled)

53. (canceled)

54. A method of delivering an agent to a cell comprising contacting the cell with the nanoparticle of claim 1.

55. The method of claim 54, wherein the agent comprises a diagnostic agent.

56. The method of claim 54, wherein the agent comprises a therapeutic agent.

57. (canceled)

58. (canceled)

59. (canceled)

60. (canceled)

61. (canceled)

62. (canceled)

63. The method of claim 47, where the admixing occurs in an organic-aqueous two phase system.

64. The method of claim 63, further comprising separating the two phase system to obtain the nanoparticles modified with the agent in the aqueous phase.


SEQUENCE LISTING (2 sequences)

SEQ ID NO: 1 - DNA, 19 bp, Artificial Sequence
  Synthetic oligonucleotide
aaaaaaaaaaattatcact

SEQ ID NO: 2 - DNA, 19 bp, Artificial Sequence
  Synthetic oligonucleotide
aaaaaaaaaaagtgataat